(19) United States
(12) Patent Application Publication
(10) Pub. No.: US 2007/0233139 A1
(43) Pub. Date: Oct. 4, 2007

(54) Surgical Jig

(76) Inventors: Nick James Theophilius Metcalfe, Muenchen (DE); Russell Lloyd, Wiltshire (GB); David Wycliffe Murray, Cuddesdon (GB)

Correspondence Address: HARNESS, DICKEY & PIERCE, P.L.C., P.O. BOX 828, BLOOMFIELD HILLS, MI, 48303, US

(21) Appl. No.: 11/669,674

(22) Filed: Jan. 31, 2007

(30) Foreign Application Priority Data
Feb. 1, 2006 (GB) GB0602055.6

(51) Int. Cl. A61F 5/00

(52) U.S. Cl. 606/87

(57) ABSTRACT

A surgical jig for preparing a distal end of a femur during an orthopedic procedure comprising an engagement surface that accommodates at least a part of at least one of a patella and patella tendon.

### CROSS-REFERENCE TO RELATED APPLICATIONS

This application claims the benefit of United Kingdom application number GB0602055.6, filed Feb. 1, 2006. The disclosure of the above application is incorporated herein by reference.

### FIELD

The present teachings relate to surgical jigs. In particular, the present teachings relate to surgical jigs for preparing a distal end of a femur.

### BACKGROUND

The statements in this section merely provide background information related to the present teachings and may not constitute prior art.

When a knee joint becomes damaged or diseased, it is known to replace all or part of the knee joint with a prosthesis. A common form of prosthesis comprises a femoral component, which is attached to a distal end of a femur, and a tibial component, which is attached to a proximal end of a tibia. The femoral and tibial components may articulate directly or may be separated by a meniscal bearing component. The femoral component also articulates with a patella, which is secured in position by a quadriceps tendon and a patellar ligament.

The articulation of a natural knee joint is stabilized by the action of medial and lateral collateral ligaments and anterior and posterior cruciate ligaments. Where possible, all of these ligaments are retained when a prosthesis is implanted, although in practice it is often necessary to remove at least the posterior cruciate ligament. It is desirable for tension in the knee ligaments after surgery to be balanced throughout the range of motion of the knee.

The most complex component of a knee prosthesis is the femoral component, since it carries not only the condylar bearing surfaces, but also the patella bearing surface, which extends along an anterior face of the distal femur. Conventional femoral components require resection of the distal end surface of the femur and the anterior and posterior faces of the femur. They also usually require two chamfered cuts to be made at the distal end of the femur anteriorly and posteriorly. The correct positioning of the femoral cuts is vitally important to ensure equal tension in the ligaments after surgery.

Conventional jigs for resecting the femur use as a reference an intramedullary rod. The cutting jig is mounted on the rod adjacent the resected femoral surface and may be moved in the anterior/posterior direction relative to the rod. In order to mount a conventional jig adjacent the distal surface of a femur, it is necessary to move the patella from its normal positional. The patella is either everted or subluxed in order to provide sufficient space for the jig. Once the jig is in the desired position, it is secured to the bone and the necessary cuts are made. The anterior/posterior position of the femoral cuts, and hence of the cutting jig, is vital in order to restore proper functioning of the knee and balance to the ligaments. Conventional jigs are provided in a range of sizes (usually five or six) in order to accommodate the range of knee sizes encountered.

Balancing of the knee ligaments during surgery conventionally takes place in three stages. First, after the distal surface of the femur and the proximal surface of the tibia have been resected, the knee is placed in full extension and a spacer block is used to measure the gap between the bones. The ligaments are balanced with the knee in extension to achieve a rectangular gap between the adjacent bone surfaces and equal tension in the collateral ligaments. Then, with the knee in 90 degrees of flexion and the femoral cutting jig attached, the spacer is again inserted, this time between the proximal tibial surface and the posterior surface of the cutting jig. The aim is to achieve the same rectangular gap and equally tensioned collateral ligaments with the knee in 90 degrees of flexion as in extension.

Balancing of the joint at this stage is complicated by the position of the patella. The quadriceps mechanism exerts a large force on the knee joint via the patella and the patella tendon. This force usually acts within the plane of articulation of the joint. However, with the patella either everted or subluxed to allow space for the femoral cutting jig, this force acts to skew the joint either laterally or medially. Correct balancing of the collateral ligaments at this stage is therefore extremely difficult.

Finally, after the anterior and posterior resections have been performed, trial prosthesis components are attached to the femur and tibia and a trial reduction is performed. Only at this point can the tension of the ligaments be checked throughout the range of motion of the knee.

### SUMMARY

The present teachings provide for a surgical jig for preparing a distal end of a femur during an orthopedic procedure comprising an engagement surface that accommodates at least a part of at least one of a patella and patella tendon.

The present teachings further provide for a surgical jig for preparing a distal end of a femur during an orthopedic procedure comprising a guide member, an engagement surface, an alignment member, and an adjustment system. The guide member has at least one tool guide. The engagement surface of the guide member accommodates at least a part of at least one of a patella and a patella tendon. The alignment member engages the guide member and the femur. The adjustment system moves the guide member relative to the alignment member.

The present teachings also provide for a method of preparing a distal end of a femur, which articulates with a proximal end of a tibia and with a patella, using a jig having an alignment member in cooperation with a guide member. The method includes: resecting a distal surface of the femur; inserting the alignment member of the jig into the femur until a proximal surface of the guide member engages the resected femoral surface, a groove on the guide member being aligned in a substantially anterior/posterior direction; inserting a spacer between the proximal surface of the tibia and a posterior surface of the guide member; adjusting an adjustment system of the jig until the posterior surface of the guide member is flush against the spacer; positioning the patella in the groove of the guide member; and balancing the tension of ligaments joining the femur and the tibia.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of a surgical jig for use on the left knee of a patient;

FIG. 2 is another perspective view of the jig of FIG. 1;

FIG. 3 is a perspective view of a mounting post of the jig of FIG. 1; and

FIG. 4 is a perspective view of an alignment member of the jig of FIG. 1.

### DETAILED DESCRIPTION

The following description is merely exemplary in nature and is not intended to limit the present disclosure, application, or uses.

With reference to the Figures, a surgical jig 2 comprises a guide member 4, an alignment member 6, and a reference member 8, comprising a mounting post 52 and a stylus 60. An adjusting means 10 acts between the guide member 4 and the alignment member 6 to permit relative translational movement between the guide member 4 and the alignment member 6. A plurality of tool guides 5 extend through the guide member to guide the bit of a drill (not shown).

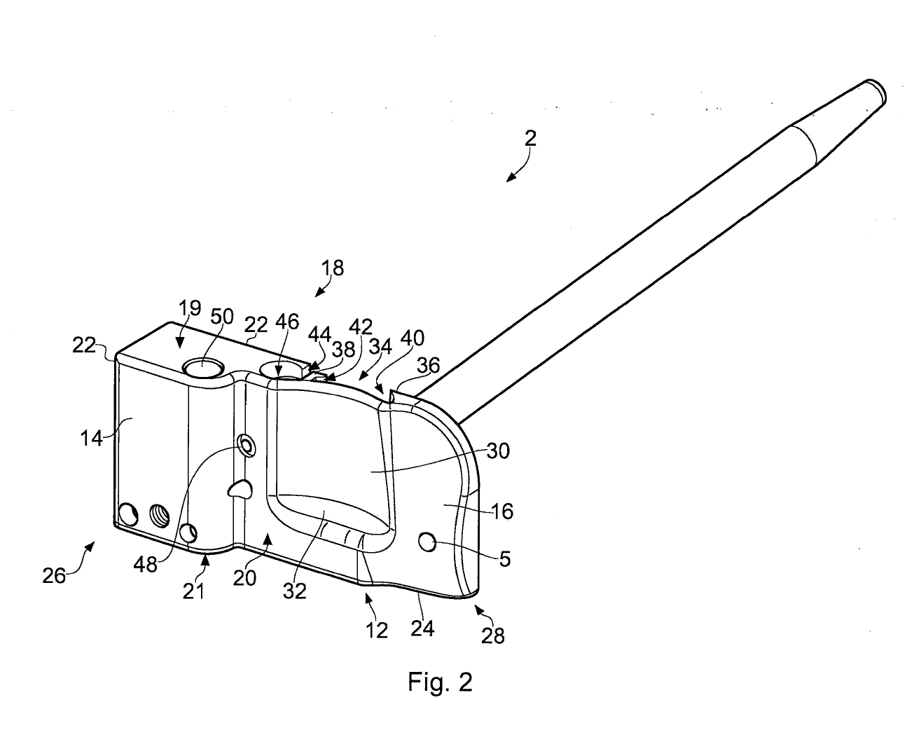

With reference particularly to FIG. 2, the guide member 4 comprises a substantially rectangular guide body 12 having a proximal surface 18, a distal surface 20, an anterior surface 19 and a posterior surface 21. The proximal and distal surfaces 18, 20 terminate at upper, anterior edges 22 and lower, posterior edges 24.

The guide body 12 is divided into a mounting portion 14 and a guide portion 16. The mounting portion 14 is disposed on the medial side 26 of the jig 2 and the guide portion 16 is disposed on the lateral side 28 of the jig 2. In the illustrated embodiment, the medial side is to the left of the jig 2, as viewed from the distal approach, but it will be appreciated that in a jig for use on the right knee of a patient, the medial side will be to the right side of the jig when viewed from a distal approach. The distal surface 20 of the guide portion 16 includes a longitudinal groove 30 that runs in a substantially anterior/posterior direction. In the illustrated embodiment, the groove 30 extends from the anterior edge 22 of the distal surface 20 and terminates adjacent to the posterior edge 24 of the distal surface 20, thus defining a seat 32. However, the groove 30 may extend the length of the guide body 12, terminating at the posterior edge 24 of the distal surface 20.

A recess 34 extends across the proximal surface 18 of the guide portion 16, opposite to the groove 30 in the distal surface 20. The recess 34 extends the length of the proximal surface 18 from the anterior edge 22 to the posterior edge 24. The recess 34 is defined by lateral and medial ridges 36, 38 that protrude from the proximal surface 18 of the guide portion 16.

The ridges 36, 38 curve toward each other, defining respective grooves 40, 42 at opposite sides of the recess 34. A longitudinal slot 44 extends into the guide body 12 from the proximal surface 18 adjacent the medial ridge 38 and parallel to the recess 34. The slot 44 is integral with a first cylindrical bore 46 that also extends the length of the guide body 12.

An adjusting screw (not shown) is located in the cylindrical bore 46 such that threaded portions of the screw protrude into the slot 44. The adjusting screw is waisted between the threaded portions to form an annular groove in a central region of the adjusting screw. A grub screw 48 extends through the guide body 12, engaging the groove of the adjusting screw and holding the adjusting screw captive within the cylindrical bore 46, such that relative translational movement between the adjusting screw and the guide body 12 is prevented.

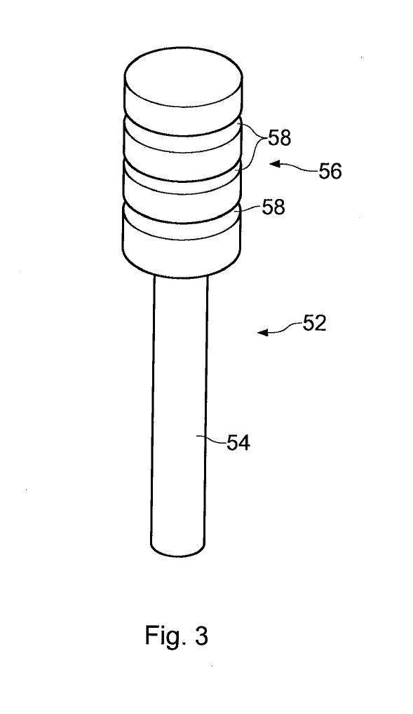

A second cylindrical bore 50 extends through the mounting portion 14 of the guide body 12. A mounting post 52, as shown in FIG. 3, is received within the second cylindrical bore 50. The mounting post 52 comprises a posterior section 54, which is received within the second cylindrical bore 50, and an anterior section 56, which is of greater diameter than the posterior section 54. A plurality of annular grooves 58 extends around the anterior section 56. A stylus 60 is received within any one of the groves 58.

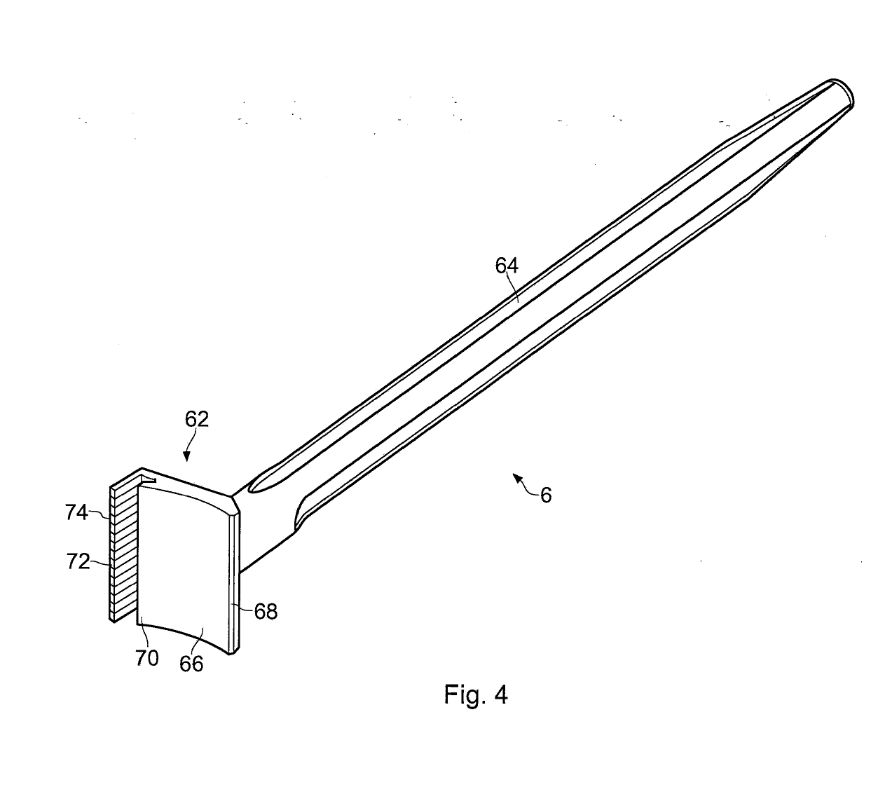

With reference particularly to FIG. 4, the alignment member 6 comprises an attachment portion 62 and an intramedullary (IM) rod 64. The attachment portion 62 comprises a body 66, lateral and medial projections 68 and 70 and an engagement arm 72. The projections 68, 70 and the engagement arm 72 each extend the length of the body 66 in a substantially anterior/posterior direction. The engagement arm 72 carries on its medial face a thread 74, which may be a rack. The IM rod 64 is formed integrally with the attachment portion 62 and extends from a proximal side of the body 66 of the attachment portion 62 at an angle that is chosen to replicate the natural valgus angle of the average patient. A range of alignment members may be provided, each having a different angle formed between the IM rod 64 and the body 66 of the attachment portion 62. An appropriate alignment member may then be selected according to the requirements of a particular patient.

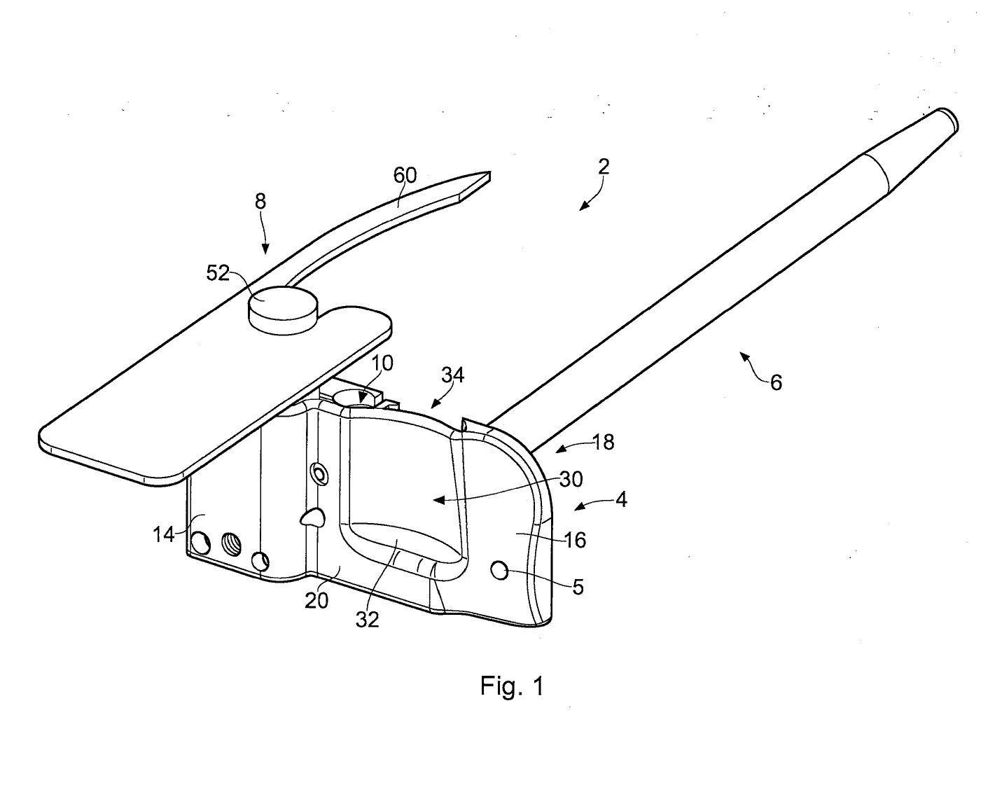

In an assembled condition of the jig, as illustrated in FIGS. 1 and 2, the attachment portion 62 of the alignment member 6 is received within the recess 34 of the guide body 12. The lateral and medial projections 68, 70 are received within the lateral and medial grooves 40, 42 of the recess 34. The engagement arm 72 is received within the slot 44 such that the rack 74 on the engagement arm 72 protrudes into the first cylindrical bore 46. The rack 74 engages the threaded portions of the adjusting screw (not shown).

The adjusting screw is prevented from translational movement relative to the guide body 12 by the interaction of the grub screw 48, the guide body 12 and the annular groove on the adjusting screw. Rotation of the adjusting screw therefore causes both the adjusting screw and the guide body 12 to be moved along the rack 74 of the engagement arm 72, thus translating the guide body 12 in the anterior/posterior direction relative to the alignment member 6. Rotation of the adjusting screw is effected by means of an Allen key or other device.

In use, the jig 2 may be employed in conjunction with a plurality of differently sized cutting blocks. The cutting blocks are sized according to the corresponding size of femoral prosthesis, and include guide portions for guiding a saw in making the required anterior, posterior and chamfered cuts to the distal end of the femur.

Prior to use, the jig 2 is placed in an assembled condition and the stylus 60 is removed. Following standard proximal tibial resection and distal femoral resection, the knee is placed in extension and an appropriately sized spacer is selected, permitting tension of the relevant soft tissues to be checked. The knee is then placed in 90° of flexion and the IM rod 64 of the jig 2 is inserted into the medullary canal of the femur until the proximal surface 18 of the guide body 12 is flush with the resected distal surface of the femur.

The selected spacer is introduced into the gap between the posterior surface 21 of the guide body 12 and the resected tibial surface. The adjusting screw is rotated causing the guide body 12 to translate relative to the IM rod 64, and hence the femur, until the posterior surface 21 of the guide body 12 is flush with the spacer. The stylus 60 is attached to a selected one of the annular grooves 58 on the mounting post 52 such that an end of the stylus 60 is adjacent the anterior femoral cortex. The anterior/posterior position of the guide body 12 is then finely adjusted by rotation of the adjusting screw until the end of the stylus 60 is in contact with the anterior femoral cortex.

Prior to fixing the position of the jig 2 with respect to the femur, a partial reconstruction of the knee is effected. The patella is returned from its everted or subluxed position and placed to rest in the groove 30 of the jig 2. The tension of the soft tissues may then be checked with the force exerted by the quadriceps mechanism acting in its correct anatomical direction. Further, the tension of the soft tissues may be checked throughout the range of motion of the knee with the jig 2 still in place, as the patella is able to track within the groove 30. Fine adjustment of the position of the jig may be conducted as necessary.

Once the correct position of the jig has been ascertained, guide holes are drilled through the guides 5 of the jig 2 and into the resected femoral surface. The jig 2 may then be removed and replaced with an appropriately sized cutting block. The size of cutting block may be selected to correspond with the annular groove 58 on the which the stylus 60 was mounted. The cutting block is attached to the femur using the guide holes drilled through the guides 5 in the jig. Anterior, posterior and chamfered cuts may then be made in the standard manner.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A surgical jig for preparing a distal end of a femur during an orthopedic procedure comprising an engagement surface that accommodates at least a part of at least one of a patella and patella tendon.

2. The surgical jig of claim 1, wherein said engagement surface includes a groove.

3. The surgical jig of claim 2 further comprising a guide member, an alignment member, and an adjustment system that moves said guide member relative to said alignment member.

4. A surgical jig for preparing a distal end of a femur during an orthopedic procedure comprising:
a guide member having at least one tool guide;
an engagement surface of said guide member that accommodates at least a part of at least one of a patella and a patella tendon;
an alignment member that engages said guide member and the femur; and
an adjustment system that moves said guide member relative to said alignment member.

5. The surgical jig of claim 4, wherein said alignment member includes an attachment portion and an intramedullary rod.

6. The surgical jig of claim 5, wherein said attachment portion includes a first engagement feature that cooperates with a second engagement feature on a proximal side of the guide member to secure said guide member to said alignment member.

7. The surgical jig of claim 6, wherein said attachment portion further includes projections that cooperate with grooves on a proximal side of said guide member.

8. The surgical jig of claim 4, wherein said adjustment system includes a threaded worm that is received within said guide member.

9. The surgical jig of claim 8, wherein said worm engages a cooperating thread on said alignment member.

10. The surgical jig of claim 8, wherein said worm includes a cylindrical member having at least one recessed portion.

11. The surgical jig of claim 10, wherein said adjustment system further includes a holding pin that engages said guide member and said recessed portion of said worm to prevent relative translation between said guide member and said worm.

12. The surgical jig of claim 4, wherein said tool guide includes a guide hole for a drill bit.

13. The surgical jig of claim 4, wherein said engagement surface further comprises a recess that extends across a distal face of said jig in a substantially anterior/posterior direction.

14. The surgical jig of claim 4, wherein said engagement surface further comprises a recess that extends across a distal face of the guide member in a substantially anterior/posterior direction.

15. The surgical jig of claim 13, wherein said recess is dimensioned to accommodate at least a part of a patella and a patella tendon.

16. The surgical jig of claim 4, further comprising a reference member.

17. The surgical jig of claim 16, wherein said reference member includes a stylus and a post.

18. The surgical jig of claim 17, wherein said stylus is adapted to reference said jig with the anterior cortex of the distal femur.

19. The surgical jig of claim 17, wherein said guide member further comprises a cylindrical opening.

20. The surgical jig of claim 19, wherein said post extends through said cylindrical opening parallel to a groove in said guide member.

21. The surgical jig of claim 20, wherein an anterior end of said post includes a plurality of annular grooves.

22. The surgical jig of claim 21, wherein said stylus is sized to be received in any one of said annular grooves of said mounting post.

23. A method of preparing a distal end of a femur, which articulates with a proximal end of a tibia and with a patella, using a jig having an alignment member in cooperation with a guide member, the method comprising:
resecting a distal surface of the femur;
inserting the alignment member of the jig into the femur until a proximal surface of the guide member engages the resected femoral surface, a groove on the guide member is aligned in a substantially anterior/posterior direction;
inserting a spacer between the proximal surface of the tibia and a posterior surface of the guide member;
adjusting an adjustment system of the jig until the posterior surface of the guide member is flush against the spacer;
positioning the patella in the groove of the guide member; and
balancing the tension of ligaments joining the femur and the tibia.

24. The method of claim 23 further comprising inserting a mounting post into a bore of the guide member and attaching a stylus to a selected one of a plurality of annular grooves of the mounting post such that a free end of the stylus is adjacent to a surface of an anterior femoral cortex; and
adjusting the adjustment system so that a free end of the stylus is in contact with the anterior femoral cortex.

25. The method of claim 24 further comprising:
drilling a reference hole in the resected femoral surface through a guide hole of the guide member;
removing the jig from the femur;
selecting an appropriately sized cutting block at least partially based on which one of the plurality of grooves the stylus is mounted to; and
securing the cutting block to the distal femur using the reference holes.

26. The method of claim 23, wherein inserting the alignment member of the jig into the femur includes inserting an intramedullary rod into a medullary canal of the femur.

27. The method of claim 23, wherein adjusting the adjustment system includes rotating a threaded worm using a key.
